(19) United States
(12) Patent Application Publication
(10) Pub. No.: US 2014/0248679 A1
(43) Pub. Date: Sep. 4, 2014

(54) Apparatus and Methods to Enhance Field Gradient For Magnetic Rare Cell Separation

(71) Applicants: Jing Zhang, Los Altos, CA (US); Glenn Yaguang Deng, San Jose, CA (US)

(72) Inventors: Jing Zhang, Los Altos, CA (US); Glenn Yaguang Deng, San Jose, CA (US)

(21) Appl. No.: 14/192,875

(22) Filed: Feb. 28, 2014

Related U.S. Application Data
(60) Provisional application No. 61/771,802, filed on Mar. 2, 2013.

(51) Int. Cl. C12N 13/00

(52) U.S. Cl. CPC C12N 13/00
USPC 435/173.9; 435/283.1

(57) ABSTRACT

The present invention discloses apparatus and methods to enhance magnetic field gradient for magnetic cell separation. One preferred embodiment in accordance with the invention comprises an electromagnet with an open gap formed between two pole tips and two driving coils with independently controlled electrical current magnitude and direction. In the specific case when two magnetic pole tips emanate magnetic fields with different magnitude and in opposite direction, the field gradient, and therefore the magnetic force exerted on MNPs, is advantageously enhanced. Spatial uniformity of the magnetic field gradient is also enhanced. Preferred embodiments are capable of magnetic cell separation for both positive and negative cell selection, as well as in-vitro capture and separation in miniaturized form of embodiments.

### CROSS-REFERENCE TO RELATED APPLICATIONS

The present application is related to U.S. provisional patent application Ser. No. 61/771,802, filed on Mar. 2, 2013, by ZHANG et al., which is included by reference herein and for which benefit of the priority date is hereby claimed.

### STATEMENT REGARDING FEDERALLY SPONSORED RESEARCH OR DEVELOPMENT

None

### FIELD OF THE INVENTION

The present invention relates to apparatus and methods for rare cell separation and biological analyses using biological samples, and more specifically, apparatus and methods to enhance effectiveness of rare cell separation using magnetic fields and magnetic nano-particles (MNPs).

### BACKGROUND

Rare cell separation is of utmost importance in experimental biology and medicine. The rapid development and progress in cell separation methods have been driven by both academic interest and practical clinical needs, catalyzed by ever increasing demands for sensitivity and selectivity with this process.

Some diseases are related to specific type of cells (or target cells) which can be used as a biomarker for diagnosing and monitoring the progression of a particular disease. For example, circulating tumor cells (CTCs) can be present in blood stream of carcinoma (or cancer) patients. Detection of CTCs is of profound clinical and scientific interest. Metastasis, the process of carcinoma tumor cells spreading and growth from a primary carcinoma tumor site to a distant secondary site, is a significant problem in carcinoma research and treatment. Recently, it has been found that CTCs are present in patients with various metastatic carcinomas. It is also beneficial for evaluation of a patient's response to therapy, especially for those patients with metastatic carcinoma. The number of CTCs in the blood has been shown to correspond to the clinical course of the disease and is a predictor of patient's overall survival rate. Some clinical studies have demonstrated correlation between CTCs counts and progression of disease for certain types of carcinoma, such as metastatic breast cancer, colorectal cancer, and prostate cancer. Better understanding of CTCs properties is expected to revolutionize carcinoma treatment. There is therefore a strong need to develop technologies to enable biological studies with rare cells such as CTCs.

However, understanding of CTCs properties is limited in both clinical and medical research since CTCs are rare cells which are difficult to be separated from their host samples. Cell separation devices are helpful and, in many cases, even mandatory for advance in related areas, including, but not limited to, life science, pharmaceutical research, and medical practice. Separated cells, after enrichment, isolation, and purification, can be used in various subsequent downstream tests and measurements, including, but not limited to, cell counts, analysis of DNA mutation and RNA/protein expression even on single cell level, study of tumor formation mechanism and metastatic processes, detection and monitoring of various diseases, performing pathology analyses, authenticating a person's identity, and classifying animal species.

Tremendous efforts have been made in developing methods to capture and separate rare biological cells in a complex suspension of cells. One of the most commonly used methods is magnetic cell separation, also known as immunomagnetic cell enrichment, where magnetic nano-particles (MNPs), also referred to as magnetic beads, are used to separate target cells from various specimen including blood, tumor tissues, biopsies, or bone marrow, etc. Such methods rely on separation of target cells by specific antibodies conjugated to MNPs which are mixed with specimen. Target cells are captured in the presence of an applied magnetic field. Captured cells are then cleaned by washing them in certain liquid solution to remove non-specific cells and unwanted materials. Finally, target cells are released or separated in order to be collected for further downstream analysis as aforementioned.

An important advantage of magnetic cell separation over other affinity-based separation methods is its capability to collect magnetically-tagged target cells and to control their movement within fluidic systems. This capability provides tremendous flexibility in downstream sub-cellular level analysis and utilization of cells for specific applications.

Magnetic nanoparticles (MNPs) are a valuable class of nano-materials with unique properties which are distinct from those of their bulk counterparts. In general, when MNPs size or diameter descends from micron range to tens of nm range, depending on specific material composition, the magnetic state migrates from multi-domain state, through single-domain state, and eventually to the super-paramagnetism regime.

One class of MNPs is of particular interest. The total magnetic energy barrier is proportional to the product of the volume of MNPs and the magnetic anisotropy (or stiffness). When the magnetic energy barrier of MNPs is on the same order of magnitude as the thermal energy, MNPs become super-paramagnetic. An extremely important and useful characteristic of super-paramagnetic MNPs is that they exhibit maximum magnetic moment in the presence of a small applied magnetic field. Upon removal of the field, they tend to exhibit zero net magnetic moment over time since their magnetic moment can be constantly disturbed and randomly flipped due to thermal energy. In this condition, MNPs exhibit low coercivity, that is, low resistance to magnetic moment reversal. This unique property makes MNPs of small size (diameter in tens of nanometers) highly manipulatable with an applied magnetic field. Zero net magnetic moment over time in the absence of external magnetic field also offers the advantage of reduced risk of particle aggregation [1].

In the case that size of MNPs is above the limit of super-paramagnetism regime, MNPs can exhibit either single-domain or multi-domain state in the absence of applied magnetic fields, depending on the particle size and magnetic property of the material. To be specific, when the particle size is larger than the characteristic exchange length of the material, MNPs exhibit multi-domain state, in which overall magneto-static energy is minimized. In another word, different portions of MNPs carry magnetic moment in different directions. On the other hand, when the particle size is smaller than the characteristic exchange length of the material, MNPs exhibit single-domain state, that is, all portions of MNPs carry magnetic moment in the same direction.

In the presence of applied magnetic field, MNPs originally in the multi-domain state can transform into the single-domain state with magnetic moment aligned with the magnetic field direction, and MNPs originally in the single-domain state reorient their magnetic moment along the field direction. In either case, the magnetic field strength is required to be large enough to overcome energy barrier associated with either demagnetization field or intrinsic magnetic stiffness of the material. Therefore, sufficient magnetic field strength is required for successful magnetic cell separation.

The magnetic force exerted on MNPs directly contributes to effectiveness of magnetic cell separation and is governed by the following equation,

F∝V*M*∇H (1)

where V is the active volume of MNPs (excluding non-magnetic materials enclosing MNPs), and M is the magnetization of the magnetic materials, or net magnetic moment per unit volume. Therefore, V*M actually denotes total magnetic moment of MNPs. ∇H is the gradient of the magnetic field, that is, the rate of magnetic field strength change versus linear spacing from the source of the magnetic field. It should be noted that once M is saturated with sufficient magnetic field strength H, further increasing H no longer contributes to increase of magnetic force on MNPs. Instead, field gradient ∇H becomes the dominant term to enhance magnetic force.

A well-designed magnetic cell separation apparatus should be flexible to work with MNPs of various sizes and material properties in order to accommodate a wide range of applications. Depending on MNPs size and material properties, apparatus to generate magnetic field for magnetic cell separation should have either sufficient magnetic field gradient to maximize magnetic force in the case of MNPs exhibiting super-magnetism or single-domain behaviors, or optimized magnetic field strength to ensure complete saturation of MNPs otherwise. It should be emphasized that, for MNPs exhibiting super-magnetism or single-domain behaviors, field gradient plays a more critical role than magnetic field strength itself and should be maximized to enhance effectiveness of magnetic cell separation, as indicated by Equation (1).

One main advantage of small size MNPs is their large effective surface areas for easy bonding to target cells and low sedimentation rates with enhanced stability [2]. Another advantage of small MNPs is that the magnetic dipole-dipole interaction between MNPs is significantly reduced since it scales as inverse of r̂6, where r is the radius of MNPs [3].

MNPs can have size ranging from nanometers up to microns, such that their dimension is smaller than or comparable to those of biological entities of interest, such as cells (10-100 micron), virus (20±500 nm), proteins (5-50 nm), or even genes (2 nm wide and 10±100 nm long). In the specific case of cells, such as CTCs, multiple MNPs can be bonded to the target cell surface. Total magnetic force exerted on multiple MNPs, and therefore total attraction force on targets cells for effective separation, can be maximized. In addition, smaller MNPs size also minimizes disturbance or even damage to target cells and simplifies downstream analyses including cell identification.

### DESCRIPTION OF RELATED ART

Tremendous efforts have been made in developing methods to capture and separate rare biological cells. As one of the most commonly used methods for cell separation and purification, magnetic cell separation relies on the tagging, or labeling, of a specific cell population in a heterogeneous mixture with magnetic nano-particles (MNPs). These magnetic tags can be tailored to target specific antigens, enabling magnetic capture of these entities with an applied magnetic field. This technique can be used to separate and enrich target cells from various samples, including blood, tumor tissues, biopsies, or bone marrow etc., for further downstream analysis.

As one specific example, CTCs are extremely difficult to detect using conventional blood analysis methods and have been difficult to separate until recently. Using advanced commercial magnetic cell separation equipment such as CellSearch system (by Johnson & Johnson), it was shown that CTCs are consistently present in the blood stream of carcinoma patients. Scientists further demonstrated that CTCs have significant clinical values as prognostic markers using the CellSearch system [4]. However, detection of CTCs still has not been adopted in routine clinical practice by American Society of Clinical Oncology (ASCO) guideline since greater sensitivity is needed to detect CTCs and more downstream analysis of CTCs are required to better understand their properties.

In addition to CellSearch system, there are additional magnetic enrichment technologies being developed. For example, techniques described in U.S. Pat. No. 3,970,518, by Giaever et al, entitled “Magnetic Separation of Biological Particles”, U.S. Pat. No. 5,200,084, by Liberti et al, entitled “Apparatus and Methods for Magnetic Separation”, U.S. Pat. No. 5,837,144, by Bienhaus et al, entitled “Method of Magnetically Separating Liquid Components”, U.S. Pat. No. 8,071,395, by Davis, entitled “Method and Apparatus for Magnetic Separation of Cells”, and techniques utilized by various commercial products including AutoMACS separation (Miltenyi Biotec), Ariol (Microsystems), RoboSep (StemCell), and MagSweeper [5]. Using these technologies and products, EpCAM or Cytokeratin positive cells can be enriched and thereafter detected.

Despite progress in method and apparatus for rare cell separation, many problems remain unsolved. Firstly, all aforementioned methods employ permanent magnet to generate magnetic field. Due to the nature of permanent magnets, the magnetic field generated is fixed. One problem with permanent magnets as magnetic field source is, therefore, that the magnetic field is not adjustable without physically changing the spacing between magnets and specimen in study. In the specific case of MagSweeper system, it is required to alternatively attach and detach the capture probe cap from the permanent magnet probe, in order to change the magnetic field strength generated by the magnetic probe for capturing and releasing target cells tagged with MNPs. This requirement complicates cell separation operation, severely limits the effectiveness of target cell collection, and compromises the sensitivity of cell separation devices. As a result, this greatly hinders wide-spread adoption and further development of advanced cell separation techniques.

Some approaches attempt to improve sensitivity by repeating capturing steps without releasing captured target cells. However, capturing efficiency is only marginally improved by this approach, and there is also concern with target cell damage. Other approaches attempt to improve cell capture sensitivity by replacing the capture probe cap enclosing the magnetic probe after each round of capture, wash, and release cycle. However, such procedure is cumbersome and time consuming with compromised operation efficiency.

In addition, it should also be pointed out that the saturation magnetization of the permanent magnet, which determines the maximal achievable magnetic field and field gradient, is usually compromised due to stringent requirements for coercivity, a measure of resistance to magnetization reversal. Since extremely high coercivity is required for permanent magnets, doping of non-magnetic materials is required, overall magnetization is therefore diluted.

In order to conveniently control magnetic field strength and direction without physically changing spacing and position between magnets and specimen in study, electromagnets can be used in lieu of permanent magnets. In this case, magnetic field is generated with driving coils winding around magnetic cores, which are in general made of soft magnetic materials, that is, magnetic moment of which can be easily reversed by applied magnetic field. Magnetic field strength and direction can be conveniently controlled by electrical current magnitude and direction. It is also flexible to control magnetic field by adjusting electrical current over time.

To precisely control magnetic field profile, Carpino et al. reported a Quadrupole electromagnet structure which consists of four separate electromagnets with spherically-shaped pole tips and was used for field-flow fractionation (FFF) as an analytical separation and characterization technique for macromolecules and particles [6]. The Quadrupole electromagnet generates a unique field profile with radially symmetric magnetic field which linearly increases with radial distance from the center axis, while the field gradient is constant within the aperture between pole tips. The channel for specimen flow occupies a thin annular space within the aperture, thereby avoiding the center axial region where magnetic field strength is small.

Another benefit of the electromagnet is that the magnetic field can be conveniently controlled over time with user-defined ramp-up and ramp-down rate to facilitate customized characterization. This feature could not be conveniently realized with permanent magnets with fixed magnetic field.

However, there is limitation with aforementioned electromagnet system. First of all, four pieces of electromagnets are required. This makes magnetic cell separation system bulky with increased power consumption and cost. In addition, while the system provides benefit of fixed gradient, the magnetic field strength in the center axis region is low and increases linearly with radius. Since sufficient magnetic field is required to make MNPs magnetically saturated, this limits the usable space for cell separation operation between magnetic pole pieces.

According to Equation (1), the magnetic force is proportional to both M and ∇H, where M is the magnetization of MNPs and increases with applied magnetic field. Until magnetization reaches saturation, higher magnetic field strength is needed for increased magnetic attraction force and effective separation. However, once MNPs are magnetically saturated, M no longer increases with increasing magnetic field, and magnetic attraction force is only dependent on the field gradient ∇H. Therefore, an effective magnetic separation apparatus requires sufficient magnetic field strength for saturation of MNPs magnetization, and more importantly, maximum field gradient after magnetic saturation of MNPs. In the specific case of small MNPs approaching super-paramagnetism regime, the field gradient requirement is dominant while the magnetic field strength requirement can be relaxed due to aforementioned reasons. In the case of MNPs with size above super-paramagnetism limit, the magnetic field strength must be at least sufficient to magnetically saturate MNPs. Once meeting minimal magnetic field strength requirement, field gradient then needs to be maximized.

Therefore, what is needed is apparatus and method to effectively manipulate and enhance magnetic field strength as well as magnetic field gradient. Furthermore, the apparatus and method must work effectively with a wide range of MNPs types with different sizes and magnetic properties. To be specific, the applicable range of the apparatus and method must encompass MNPs sizes in super-magnetism regime and beyond without compromising effectiveness of magnetic cell separation.

### BRIEF SUMMARY OF THE INVENTION

To overcome the limitations in the prior art described above, and to overcome other limitations which will become apparent upon reading and understanding the present specification, the present invention discloses methods and corresponding magnet designs to enhance magnetic field gradient for effective magnetic cell separation. Various embodiments of the present invention are described herein.

One preferred embodiment in accordance with the present invention is in the form of an electromagnet with one magnetic core with an open gap formed between pole tip surfaces and two separate driving coils. Electrical current magnitude and direction through two coils can be independently controlled. In the specific case when two magnetic pole tips emanate magnetic fields, in opposite direction and preferably with different magnitude, the field gradient, and therefore the magnetic force exerted on MNPs, is advantageously enhanced. The magnetic core can further comprise a removable section such that the electromagnet is reconfigurable.

In another embodiment, an electromagnet can be combined with a permanent magnet. The pole tip of the electromagnet and the pole tip of the permanent magnet form an open gap. The pole tip of the electromagnet and the permanent magnet emanates magnetic fields, in opposite direction and preferably with different magnitude, resulting in advantageously enhanced magnetic field gradient.

In yet another embodiment, the magnetic field can be generated by two separate pieces of permanent magnets. The pole tip of the first permanent magnet and that of the second permanent magnet form an open gap. The pole tip of the first permanent magnet and the pole tip of the second permanent magnet both emanate magnetic field, in opposite direction and preferably with different magnitude, resulting in advantageously enhanced magnetic field gradient.

These and other features and advantages of the invention will be apparent upon reading of the following detailed description of preferred embodiments taken in conjunction with the Figures in which like reference numerals indicate like elements throughout.

### IN THE DRAWINGS

FIG. 1 shows a typical M-H loop, that is, magnetization vs. applied magnetic field curve, of magnetic nano-particles (MNPs).

FIG. 2 shows one embodiment of electromagnets for magnetic cell separation described in prior art.

FIG. 3A illustrates a reference electromagnet consisting of one magnetic core and one driving coil. FIG. 3B shows corresponding magnetic field and field gradient profile versus spacing from the pole tip.

FIG. 4 illustrates one embodiment (Embodiment #1) in accordance with the present invention.

FIG. 5A illustrates one operation mode (Mode 1) of Embodiment #1 for enhanced magnetic field strength. FIG. 5B shows corresponding magnetic field and field gradient profile versus spacing from the pole tip.

FIG. 6A illustrates another operation mode (Mode 2) of Embodiment #1 for enhanced magnetic field gradient. FIG. 6B shows corresponding magnetic field and field gradient profile versus spacing from the pole tip.

FIG. 7 compares magnetic field and field gradient with Embodiment #1 shown in FIG. 4 versus the reference electromagnet shown in FIG. 3A.

FIG. 8 shows Embodiment #1 which further comprises a multi-section and reconfigurable magnetic core.

FIG. 9 shows another embodiment (Embodiment #2) using a hybrid configuration with an electromagnet combined with a permanent magnet.

FIG. 10 shows yet another embodiment (Embodiment #3) using two permanent magnets.

FIG. 11A and FIG. 11B shows yet another embodiment (Embodiment #4) using two separate electromagnets to enhance magnetic field strength and field gradient, respectively.

FIG. 12 shows one alternative embodiment of the open gap between pole tips for further improved magnetic field and field gradient.

FIG. 13 shows yet another alternative embodiment of the open gap.

FIG. 14 illustrates one example of methods of use for magnetic cell separation using preferred embodiments.

FIG. 15 illustrates another example of methods of use for magnetic cell separation using preferred embodiments.

FIG. 16 illustrates yet another example of methods of use with preferred embodiments miniaturized for in-vitro magnetic cell separation.

### DETAILED DESCRIPTION OF THE PREFERRED EMBODIMENTS

In the following description of the embodiments, reference is made to the accompanying drawings that form a part hereof, and in which is shown by way of illustration of the specific embodiments in which the invention may be practiced. It should be noted that the figures discussed herein are not drawn to scale and thicknesses of lines are not indicative of actual sizes. It is to be understood that other embodiments may be utilized since structural changes may be made without departing from the scope of the present invention.

Furthermore, in the following detailed description of embodiments of the present invention, numerous specific details are set forth in order to provide a thorough understanding of the present invention. However, it will be recognized by one of ordinary skill in the art that embodiments of the present invention may be practiced without these specific details. In other instances, well known methods, procedures, and components have not been described in detail as not to unnecessarily obscure aspects of the present invention.

The discussion will begin with description and overview of magnetic nano-particles (MNPs). Magnetic nanoparticles (MNPs) possess unique properties which are distinct from those of their bulk counterparts. When MNPs size or diameter descends from the micron range to the tens of nm range, depending upon specific material properties, the magnetic state of MNPs migrates from multi-domain state, through single-domain regime, and eventually to the super-paramagnetic state.

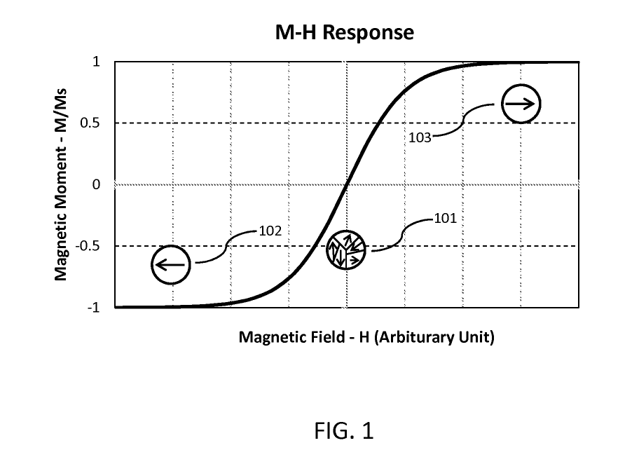

A typical M-H loop of MNPs, that is, magnetization vs. applied magnetic field curve, is shown in FIG. 1. When size of MNPs is small (on the order of tens or hundreds of nm depending on magnetic properties) and total magnetic energy barrier of MNPs is comparable to the thermal energy, MNPs become super-paramagnetic. The total magnetic energy barrier is proportional to the product of the volume of MNPs and the magnetic anisotropy (or stiffness). An extremely important and useful characteristic of super-paramagnetic MNPs is that they exhibit saturated magnetic moment 102/103 whose direction follows even a small applied magnetic field. Upon removal of the field, they tend to exhibit zero net residual magnetization when averaged over time. In another word, MNPs exhibit low coercivity, that is, low resistance to magnetic moment reversal.

In the case that size of MNPs is larger than the limit of super-paramagnetism regime, MNPs can exhibit either single-domain or multi-domain state in the absence of applied magnetic field, depending on the size of MNPs and the magnetic properties. To be specific, when the particle size is larger than characteristic exchange length of the material, MNPs exhibit multi-domain state 101, in which overall magnetic energy is minimized. In another word, different portion of MNPs can carry magnetic moment in different directions. When the particle size is smaller than characteristic exchange length of the material, MNPs exhibit single-domain state. In the presence of applied magnetic field, MNPs originally in multi-domain states transform into single-domain states with magnetic moment aligned with the field direction, while MNPs originally in single-domain states reorient their magnetic moment along the applied field direction. In either case, the magnetic field strength is required to be large enough to overcome energy barrier associated with either demagnetization field or intrinsic magnetic stiffness. In general, the higher the magnetic moment, the higher the applied magnetic field is required. For example, if MNPs saturation magnetization Ms is 500 emu/cc, or 6,300 Guass (4π*500) in SI units, the field required to saturate MNPs is on the order of 6,300 Oe (Orested).

According to Equation (1), the magnetic attraction force on MNPs is proportional to both magnetization M of MNPs and magnetic field gradient ∇H. One important implication of FIG. 1 on requirements for the magnetic field source, in the case of either electromagnet or permanent magnet, is that, the magnetic field provided must be sufficient to magnetically saturate MNPs. Once this requirement is met, magnetic field gradient plays a more critical role than the magnetic field strength itself and should be maximized. Therefore, a well-designed magnet for magnetic cell separation system should be optimized for both magnetic field, and more importantly, magnetic field gradient.

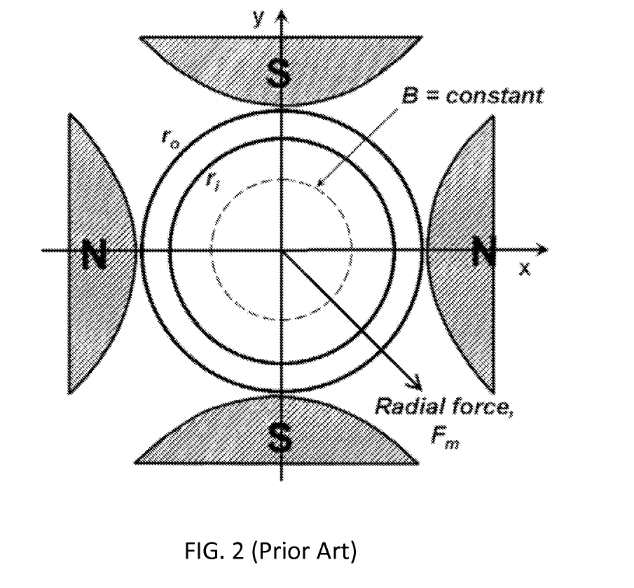

FIG. 2 shows one prior art embodiment of a magnetic field source in the form of an electromagnets for magnetic cell separation. The electromagnet apparatus employs a unique Quadrupole configuration which generates a unique and elegant field profile with radially symmetric magnetic field which linearly increases with radial distance from the center axis, while the field gradient is constant within the aperture between pole tips. The channel for specimen flow occupies a thin annular space within the space confined by four magnetic pole tips, thus avoiding the axial region where the field strength is small. The magnetic field can be conveniently enabled, disabled, and adjusted by varying current magnitude and direction through driving coils. There is also flexibility to control magnetic field over time with user-defined ramp-up and ramp-down rate to facilitate characterization needs.

However, there is limitation with aforementioned electromagnet apparatus. First of all, four pieces of electromagnets are required and make magnetic cell separation system bulky with increased power consumption and cost. In addition, while the system provides benefit of fixed gradient, the magnetic field in the center axis area is low. Since certain magnetic field strength, depending on MNPs size and magnetic properties, is required to magnetically saturate MNPs, this confines the usable space between magnetic pole pieces.

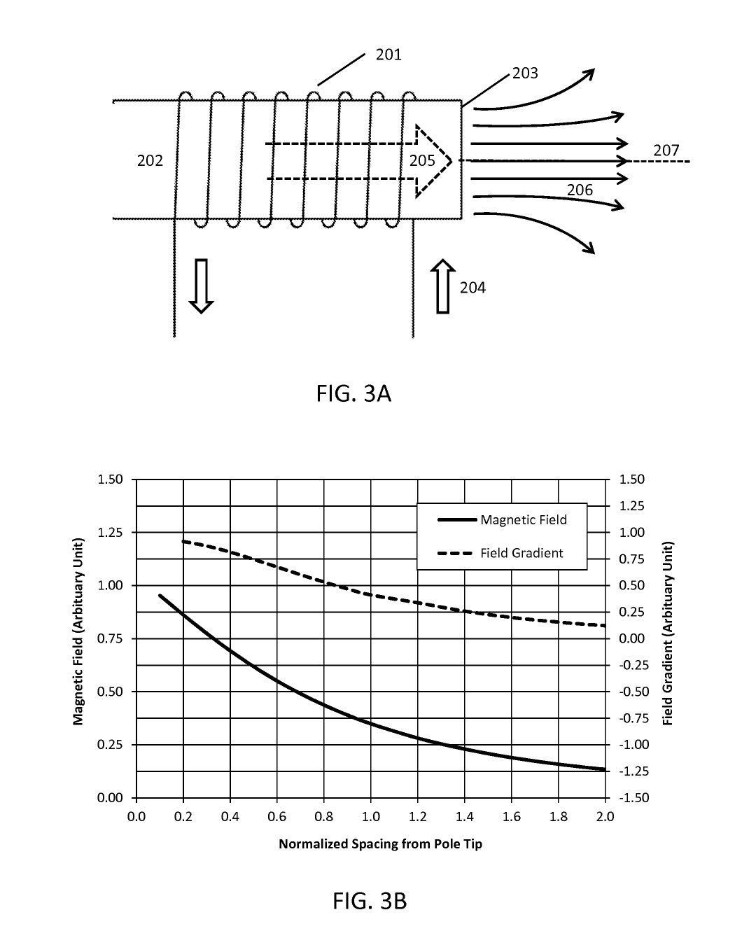

Before embodiments in accordance with the present invention are described, and for the purpose of comparison, a reference electromagnet structure, as shown in FIG. 3A, is illustrated. The electromagnet serves as a magnetic field source and consists of a magnetic core 202 and a single electrical driving coil 201. The coil structure 201 winds around the magnetic core 202 with multiple turns. When an electrical current 204 flows through the coil 201, a magnetic field is generated in the direction as governed by the right-hand rule and magnetization 205 is induced within the magnetic core 202. The boundary of the magnetic core 202 is referred to as pole tip 203. The induced magnetization 205 terminates at the surface of pole tip 203. As governed by Maxell Equations, the magnetic field emanates from the surface of pole tip 203 due to terminated magnetization 205. The magnetic field lines are illustrated by 206. It should be noted that the magnetic field also emanates from the other end of the magnetic core 202 but is not shown in the figure.

The magnetic field profile along center axis 207 is calculated and plotted in FIG. 3B. The longitudinal axis represents the spacing from the surface of the pole tip 203 and is normalized to half width of the pole tip. The vertical axis represents the magnetic field strength and field gradient. The solid line shows the magnetic field strength, while the dotted line shows the magnetic field gradient. It should be pointed out that both magnetic field strength and field gradient is highly dependent on the spacing. Either the spacing itself, or the dependence on the spacing, should be minimized to enhance effectiveness of magnetic cell separation.

### Embodiment #1

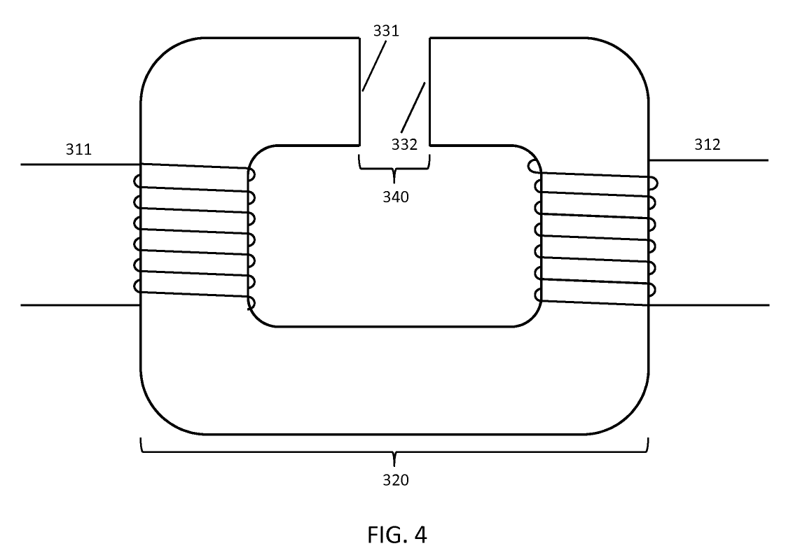

FIG. 4 illustrates one embodiment (Embodiment #1) in accordance with the present invention. The electromagnet consists of a magnetic core 320 with an open gap 340 and two driving coils 311 and 312. An open gap 340 is formed between two pole tips 331 and 332 of the magnetic core 320. Coils 311 and 312 can be independently driven by electrical currents of user-defined direction and magnitude. The purpose and advantage of two independently controlled coils will become apparent in the forthcoming description.

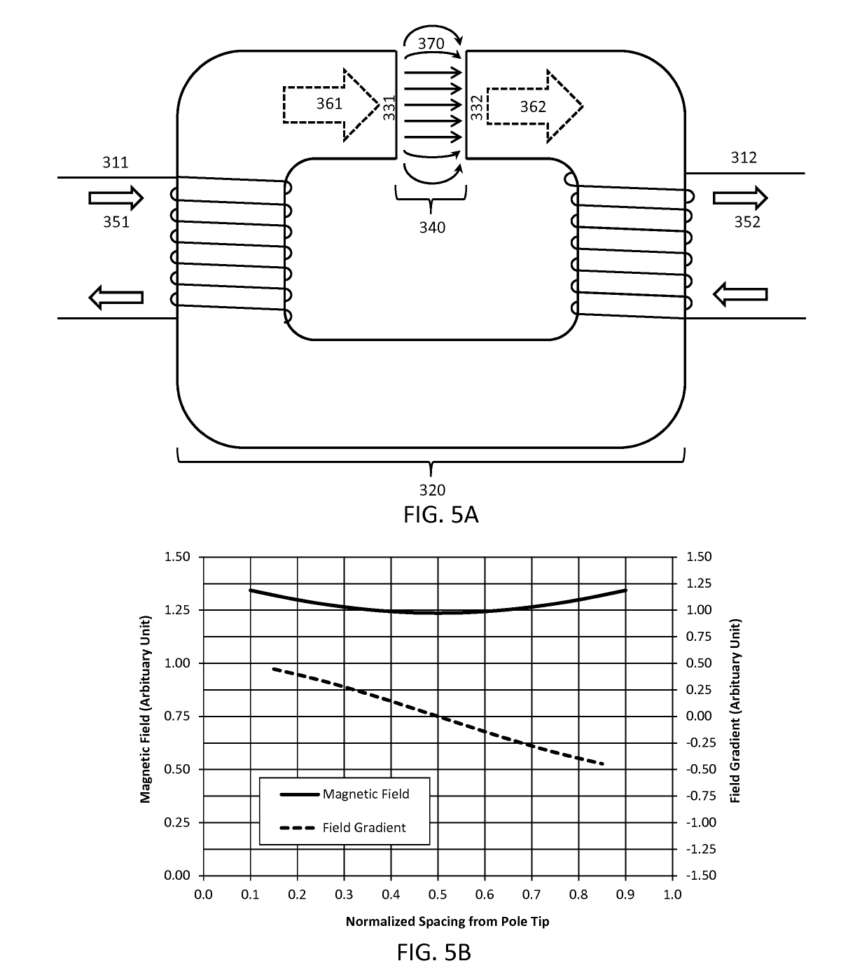

FIG. 5A illustrates one operation mode (Mode 1) of Embodiment #1 as shown in FIG. 4. In this operation mode, both coils 311 and 312 are activated with electrical current flow 351 and 352. The current flow 351 and 352 both induce magnetization in the magnetic core 320. The direction of the current flow 351 and 352 through the coils is set up such that the induced magnetization due to each current flow enhances each other. In addition, the magnitude of a first current is preferably substantially comparable to that of a second current. Induced magnetization 361 and 362 within the magnetic core 320 terminate at pole tips 331 and 332. As a result and governed by Maxwell Equations, magnetic field as shown by magnetic field lines 370 emanates in the open gap 340 between pole tips 331 and 332. The magnetic field is substantially perpendicular to the surface of pole tips 331 and 332 in the center region of the pole tip surfaces, while its direction starts to deviate from perpendicular near the edge of the pole tips. This phenomenon is known as the fringing effect. This center region with the field perpendicular to pole tip surfaces is desirable for magnetic cell separation operations and should be maximized.

The shape of the magnetic core 340 along the magnetization direction 361 and 362 is preferably C-shaped for Embodiment #1. The cross-section shape of the magnetic core 320 is preferably square, rectangular, or circular.

Specific material properties of the magnetic core 320 are required for practical electromagnet applications. Such requirements include high permeability μ and high saturation magnetization Ms. Permeability (mu) is a measure of easiness for magnetic materials to be magnetized in an applied magnetic field and is defined as the ratio between magnetization of the magnetic core vs. the applied magnetic field. Materials with high permeability are usually referred to as soft magnetic materials. High permeability is necessary to reduce electrical current needed to induce magnetization in the magnetic core and therefore effectively generate large magnetic field for a given current. Before pole tips reach magnetic saturation, the magnetic field strength H along center axis of pole tips 361 and 362 can be expressed as

H=μ*(N1*I1+N2*I2)/L (2)

where N1 and N2 are the number of turns of coils 311 and 312, respectively. I1 and I2 are the magnitude of the electrical current 351 and 352 following through coils 311 and 312, respectively. L is the length of the open gap 340 along the direction of induced magnetization 361/362, or equivalently the distance between surfaces of pole tips 331/332. It should be noted that once pole tips of the magnetic core reach saturation, the maximum magnetic field no longer increases with further increased driving current and is limited by saturation magnetization Ms of pole tip materials. Therefore, higher saturation magnetization Ms is required for the magnetic core 320, and especially pole tips 361/362.

For aforementioned reasons, both high permeability and high saturation magnetization are required. Specifically, for effective practice of embodiment #1, the magnetic core 320 should have permeability greater than 500 and the pole tips 361/362 should have saturation magnetization Ms greater than 0.5 Tesla. Examples of soft magnetic materials which meet these requirements include, but are not limited to, Permalloy, Permendur, etc. For the purpose of reference, if Ms of the pole tip material is 0.5 Tesla, maximal achievable magnetic field strength at pole tip surfaces is 5,000 Oe with two pole tips forming an open gap.

Magnetic field strength and field gradient generated by the electromagnet Embodiment #1 in the operation mode (Mode 1) as illustrated in FIG. 5A are calculated and plotted in FIG. 5B. The longitudinal axis represents the spacing from the surface of the pole tip 331 along the center axis of pole tips 361/362 and is normalized to half width of the pole tips. The vertical axis represents the magnetic field strength and the field gradient. It should be noted that for the purpose of easy comparison, the vertical axis for both the magnetic field strength and the field gradient is on the same scale as in FIG. 3B, and the magnetic property of the magnetic core 320 is the same as that of the magnetic core 202 in FIG. 3B. Furthermore, the electromotive force, N1*I1 for the coil 351 and N2*I2 for the coil 352, is identical to that of the magnetic core 202 as shown in FIG. 3B. For the purpose of calculation, it is assumed that the length of the open gap 340 is half width of the pole tips. However, it should be noted that, when normalized to half width of the pole tips, the length of the open gap 340 is preferably in the range of 0.1 to 5.0 for practical applications.

The solid line in FIG. 5B shows the magnetic field strength, while the dotted line shows the magnetic field gradient. Compared with FIG. 3B, the magnetic field strength in FIG. 5B is greatly improved. It is also noticeable that there is much less spacing dependence compared to that of an electromagnet with a single pole tip as shown in FIG. 3A and the prior art Quadrupole electromagnet as shown in FIG. 2. As for the field gradient, it is shown by the dotted line that the field gradient is maximal at both pole tips 361/362 but with opposite signs. Along the center axis from pole tip surface 361 to pole tip surface 362, there is a zero-crossing point at which the sign of field gradient reverses. In the specific case that the electromotive force N1*I1 for coil 351 is identical to N2*I2 for coil 352, this zero-crossing point is exactly in the mid-point between two pole tips 361/362. By changing the current I1 and I2, the zero-crossing point can be conveniently and advantageously controlled. The significance of this zero-crossing point control is that the direction of magnetic attraction force applied on MNPs and therefore the direction of the movement is opposite on both sides of the zero-crossing point as governed by Equation (1). It is thus feasible to manipulate MNPs movement direction by simply changing current flow I1 and I2 without mechanically moving the electromagnet or specimen in study.

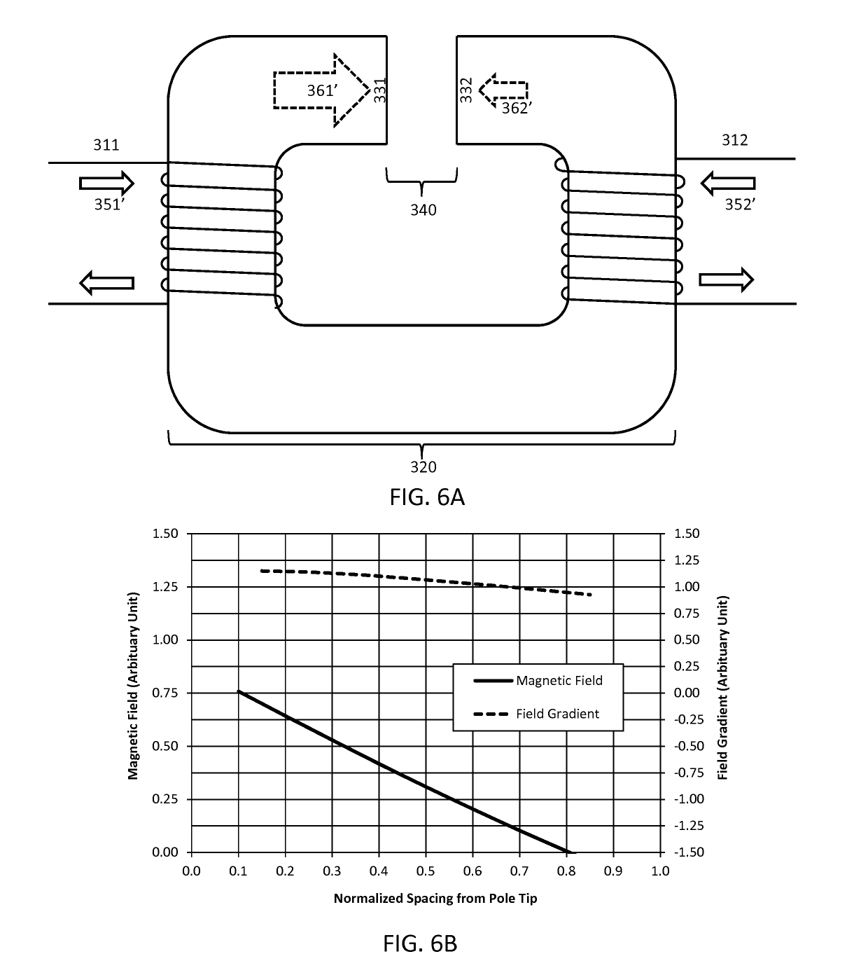

While the operation mode (Mode 1) illustrated in FIG. 5A advantageously enhances magnetic field strength with minimal spacing dependence and controls sign of field gradient within open gap for MNPs movement direction control, FIG. 6A illustrates another operation mode (Mode 2) of Embodiment #1 for enhanced magnetic field gradient. Unlike in FIG. 5A, where the direction of the current flow 351 and 352 is set up such that the induced magnetization by each current flow enhances each other, the direction of the current flow 351′ and 352′ in FIG. 6A is set up such that the induced magnetization and therefore the magnetic field generated by each current flow opposes each other. Specifically, as illustrated in FIG. 6A, current 351′ induces magnetization 361′ in a first direction, and current 352′ induces magnetization 362′ in a second direction which is opposite to the first direction. In addition, the magnitude of the current is preferably different, with a first current to generate a first magnetic field strength, and a second current which is less than the first current to generate a second magnetic field which is less than the first magnetic field in strength. Before pole tips reach magnetic saturation, the magnetic field strength H along center axis of pole tips 361/362 can be expressed as

H=μ*(N1*I1−N2*I2)/L (3)

where N1 and N2 are the number of turns of coils 311 and 312, respectively. I1 and I2 are the magnitude of the electrical current 351′ and 352′ following through coils 311 and 312, respectively. L is the length of the open gap 340 along the direction of induced magnetization 361′/362′, or equivalently the distance between surfaces of pole tips 331/332. It should be noted that, compared to Equation (2), plus ‘+’ in parenthesis is replaced with minus ‘−’ due to opposing magnetic fields generated by said first and second electrical current.

FIG. 6B shows corresponding magnetic field strength and field gradient profile versus spacing from the pole tip for the operation mode (Mode 2) as illustrated in FIG. 6A. Similar to FIG. 5B, the longitudinal axis represents the spacing from the surface of the pole tip 331 and is normalized to half width of the pole tip. The vertical axis represents the magnetic field and field gradient. For the purpose of calculation, it is assumed that the length of the open gap 340 is half width of the pole tip. It is further assumed that, for the purpose of illustration, the current 352′ induces a magnetic field which is 30% of that induced by the current 351′ but in opposite direction. The solid line in FIG. 5B shows the magnetic field strength, while the dotted line shows the magnetic field gradient. Compared with FIG. 5B and FIG. 3B, the magnetic field gradient in FIG. 6B is significantly improved. It is also noticeable that there is much less spacing dependence compared to that of an electromagnet with a single pole tip as shown in FIG. 3A and the operation mode (Mode 1) as shown in FIG. 5A.

It is apparent that the operation mode (Mode 2) shown in FIG. 6A advantageously enhances the magnetic field gradient instead of the magnetic field strength. This is especially desirable with small MNPs, which are more widely adopted than their larger counterparts due to aforementioned reasons. As already explained, the magnetic field strength required to saturate small MNPs, especially those approaching para-magnetic limit, is small due to assistance by the thermal energy. Once MNPs reach magnetic saturation, magnetic attraction force on MNPs is more dependent on the magnetic field gradient rather than the magnetic field strength.

In summary, with the same Embodiment #1 as shown in FIG. 4, but different operation modes as illustrated in FIGS. 5A and 6A, either the magnetic field strength or the magnetic field gradient can be selectively and advantageously enhanced with improved spatial uniformity and minimal spacing dependence compared with that shown in FIG. 3A/3B. This supports and facilitates the needs for magnetic cell separation application with a wide range of MNPs sizes and magnetic properties. The optimization of MNPs sizes and properties, although not subject matters of the present invention, can be engineered with greater flexibility and less constraints.

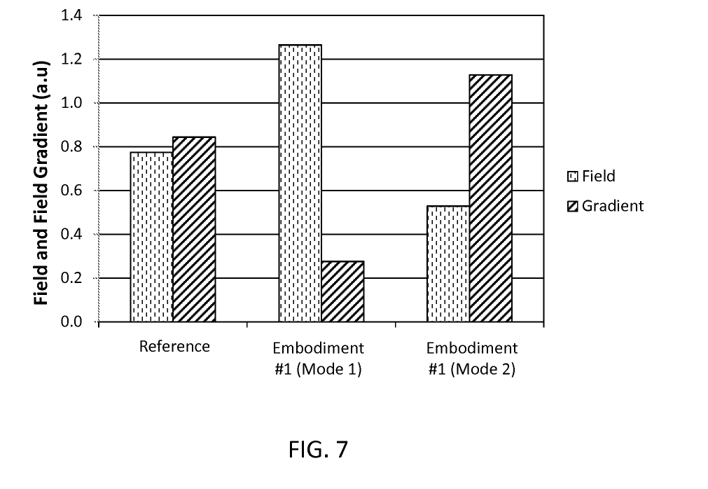

To quantify the benefit with the present invention, FIG. 7 compares magnetic field strength and field gradient with Embodiment #1 shown in FIG. 4 versus the reference electromagnet shown in FIG. 3A. For the purpose of illustration, the spacing for the field calculation is 30% when normalized to half width of the pole tip, corresponding to 0.3 on longitudinal axis in FIG. 3B/5B/6B. With the reference electromagnet shown in FIG. 3A as the baseline, the magnetic field strength is enhanced by ˜60% with Embodiment #1 in Model, and the field gradient is enhanced by ˜35% with Embodiment #1 in Mode 2. In the specific case of Embodiment #1 Mode 2, it is feasible to increase the magnetic force on MNPs by 35%, or to reduce MNPs volume by 35% without compromising magnetic separation force.

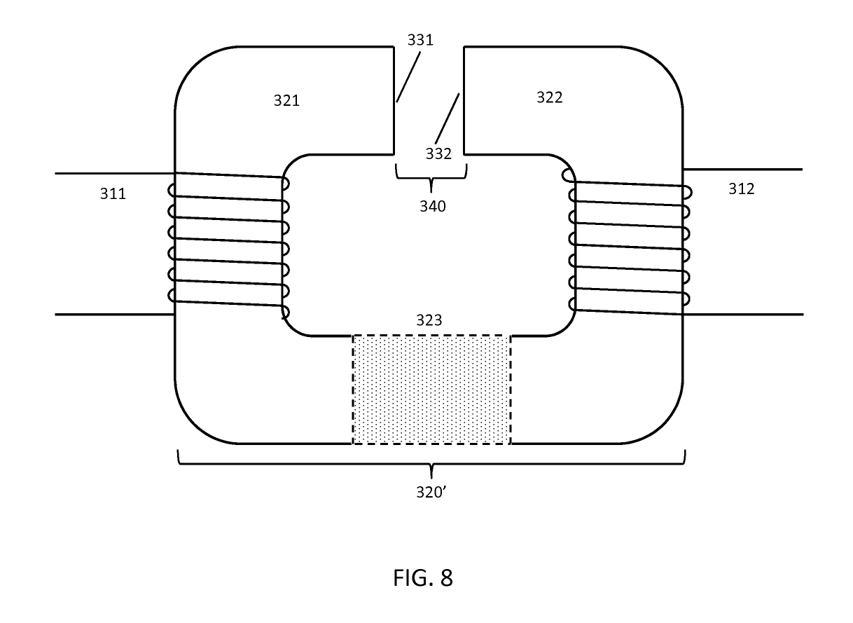

FIG. 8 shows Embodiment #1 which further comprises a multi-section and reconfigurable magnetic core. Instead of a single-piece magnetic core 320 as shown in FIG. 4, the magnetic core 320′ consists of a plurality of sections. In one particular case, the magnetic core 320′ consists of sections 321, 322, and 323. Section 323 can be further made removable. This is beneficial especially in the case of Mode 2 operation as shown in FIG. 6A when the magnetic field induced by coils 311/312 opposes each other. Removing said section 323 can reduce magnetic coupling between said sections 321/322 and improve power efficiency for generation of magnetic field. The length of the said piece 323 can be further changed so that the length of the open gap 340 is adjustable.

As stated earlier, the magnetic field is substantially perpendicular to the surface of pole tips 331 and 332 in the center region of the pole tip surfaces, while its direction starts to deviate from perpendicular at the edge of the pole tips. This fringing effect can be controlled by varying the ratio of the length of the open gap 340 versus the width of the pole tip surface 331/332. A smaller ratio leads to an increased usable center region with the field substantially perpendicular to pole tip surfaces and is desirable for magnetic cell separation, as long as there is sufficient physical space between pole tips for specimen in study.

### Embodiment #2

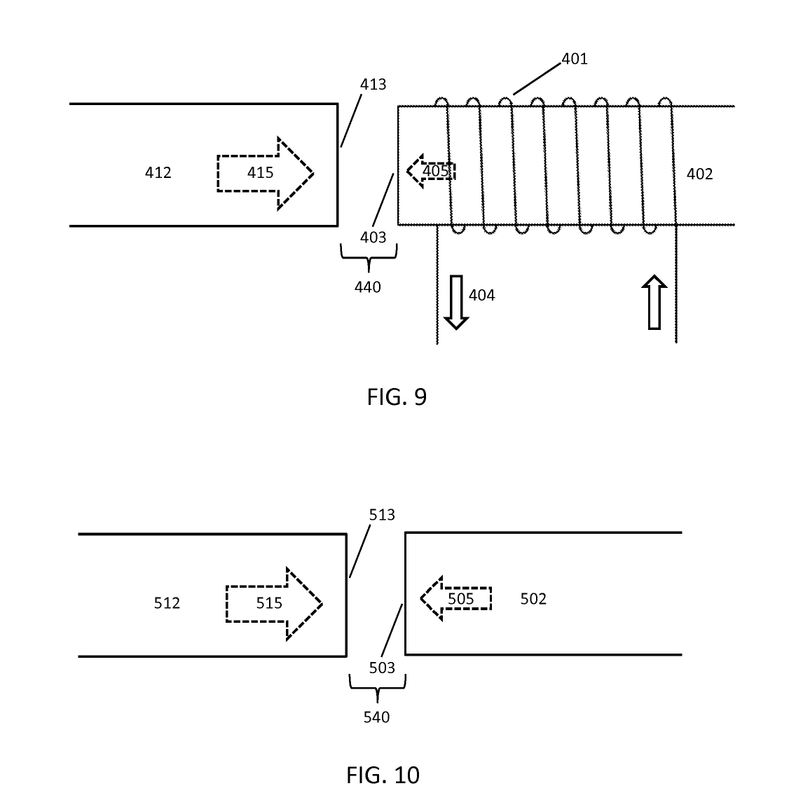

In another embodiment (Embodiment #2), an electromagnet can be combined with a permanent magnet, using the hybrid configuration as illustrated in FIG. 9. The pole tip 413 of the permanent magnet core 412 and the pole tip 403 of the electromagnet core 402 form an open gap 440. To simulate operation Mode 2 of the Embodiment #1, the magnetization 415 of the said permanent magnet core 412 and the magnetization 405 of the said electromagnet core 402 are in opposite directions. The pole tip 403 of the said electromagnet and the pole tip 413 of the said permanent magnet therefore emanate magnetic fields in opposite directions. The field generated by the said electromagnet core 402 can be controlled by adjusting driving current 404 and is preferably different from that generated by the said permanent magnet core 412, resulting in advantageously enhanced magnetic field gradient. The characteristic of the magnetic field and field gradient profile is comparable to that shown in FIG. 6B. It should be pointed out that the present embodiment can be configured to enhance magnetic field strength instead of field gradient, by reversing direction of the current flow 404 in the electromagnet coil or direction of magnetization 415 of the permanent magnet.

### Embodiment #3

In yet another embodiment (Embodiment #3), the magnetic field can be generated by two separate pieces of permanent magnets, as illustrated in FIG. 10. The pole tip 513 of the first permanent magnet 512 and the pole tip 503 of the second permanent magnet 502 form an open gap 540. To simulate operation mode 2 of the Embodiment #1, the magnetization 515 of the said first permanent magnet 512 and the magnetization 505 of the said second permanent magnet 502 are in opposite directions. The pole tip 513 of the said first permanent magnet 512 and the pole tip 503 of the said second permanent magnet 502 therefore emanate magnetic fields in opposite directions. The field generated by the second permanent magnet 502 can be controlled by selecting magnetic materials with different magnetic properties, especially saturation magnetization Ms, and is preferably less than the field generated by the first permanent magnet 512, resulting in advantageously enhanced magnetic field gradient. The characteristic of the magnetic field and field gradient profile is comparable to that shown in FIG. 6B. It should be pointed out that the present embodiment can be configured to enhance magnetic field strength instead of field gradient, by reversing magnetization 505 or 515 for one of permanent magnets.

### Embodiment #4

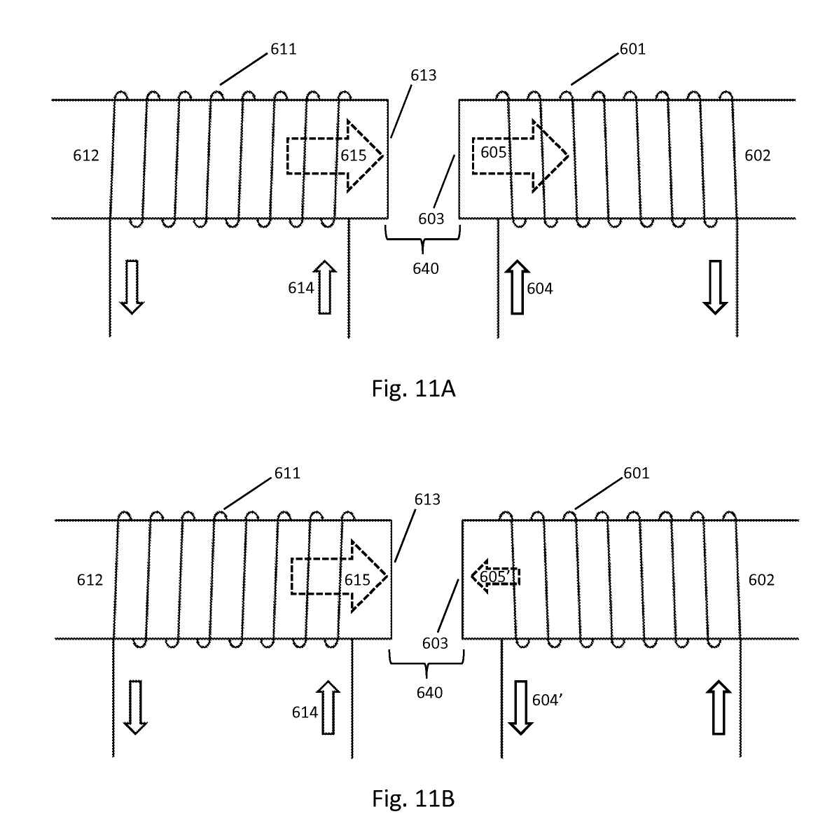

FIG. 11A and FIG. 11B shows yet another embodiment (Embodiment #4) using two separate electromagnets to enhance magnetic field and field gradient, respectively.

The operation mode of FIG. 11A is equivalent to Operation Mode 1 of Embodiment #1 as illustrated in FIG. 5A. Instead of one electromagnet, two separate electromagnets are used. The pole tip 613 of the first electromagnet core 612 and the pole tip 603 of the second electromagnet core 602 form an open gap 640. The first electromagnet comprises a magnetic core 612 and an electrical driving coil 611. When an electrical current 614 flows through the said coil 611, magnetization 615 is induced within the magnetic core 612. The induced magnetization 615 terminates at the pole tip 613. The magnetic field therefore emanates from surface of the pole tip 613. The second electromagnet comprises a magnetic core 602 and an electrical driving coil 601. When an electrical current 604 flows through the said coil 601, magnetization 605 is induced within the magnetic core 602. The induced magnetization 605 terminates at the pole tip 613. The magnetic field therefore emanates from the pole tip 603. The magnetic field emanated by said pole tips 603/613 enhances each other. The overall magnetic field strength in the open gap 640 is therefore advantageously enhanced. The characteristic of the magnetic field and field gradient profile is comparable to that shown in FIG. 5B.

The operation mode of FIG. 11B is equivalent to Operation Mode 2 of Embodiment #1 as illustrated in FIG. 6A. Compared with FIG. 11A, the electrical current flow 604′ is reversed, therefore reversing the direction of induced magnetization 605′. The fields emanating from pole tips 603/613 therefore oppose each other. This results in faster change of the magnetic field strength versus spacing in open gap 640, therefore advantageously enhancing the field gradient. The characteristic of the magnetic field and field gradient profile is comparable to that shown in FIG. 6B.

### Additional Embodiments

Magnetic field and field gradient can be further improved in addition to aforementioned embodiments. Since the magnetic field emanates from pole tips, the most effective approach for further improvement is to optimize configuration of pole tips and the open gap.

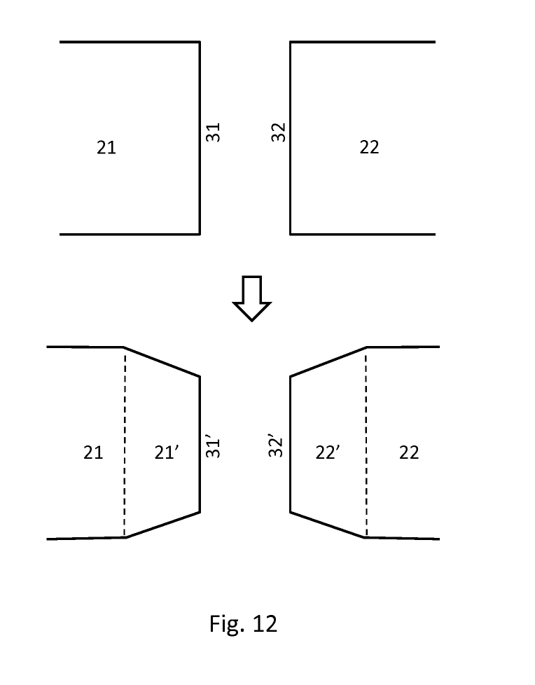

FIG. 12 shows one alternative embodiment of the open gap between pole tips. Instead of magnetic cores 21/22 with fixed cross-section area which is identical to that of pole tips 31/32, the magnetic cores include tapered sections 21′/22′ with cross-section area shrinking towards pole tips 31′/32′. In another word, cross section area of pole tips 31′/32′ is smaller than that of main magnetic cores 21/22. Total magnetic moment carried by the magnetic cores is also referred to as magnetic flux. As governed by Maxell equations, this allows the magnetic flux to be concentrated at pole tips 31′/32′. Since emanated magnet field is linearly proportional to flux density in said tapered sections 21′/22′, which is inversely proportional to the cross-section area, the magnetic field and field gradient are advantageously enhanced.

To avoid magnetic saturation of pole tips, the tapered sections 21′/22′ further consist of magnetic materials which preferably have higher saturation magnetization than that of the main magnetic cores 21/22. This allows main sections of magnetic cores 21/22 and tapered sections 21′/22′ to be optimized independently. Specifically, the tapered sections 21′/22′ require higher saturation magnetization to avoid magnetic saturation and degradation of magnetic field gradient, while the main magnetic cores 21/22 require higher permeability for efficient generation of magnetic flux. For example, Permendur has higher saturation magnetization, therefore higher flux carrying capability than that of Permalloy, and is preferred for pole tips.

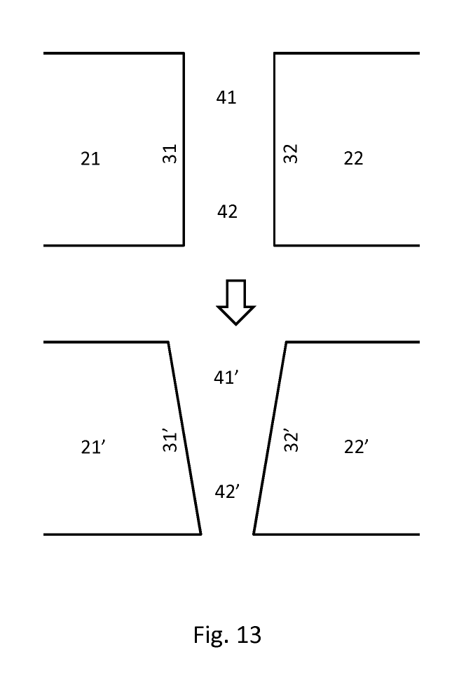

FIG. 13 shows yet another alternative embodiment of the open gap. Instead of an open gap with pole tips 31/32 in parallel, the pole tip surface 31′/32′ can be slanted with length of the open gap varying. In the specific case as shown in FIG. 13, the length of the open gap in region 41′ is larger than that of region 42′. As a result, the magnetic field and field gradient at region 41′ is lower than that of region 42′. This feature is useful for various applications with MNPs. For example, when MNPs in a fluidic flow moves from region 41′ to region 42′, it is expected that larger MNPs with higher magnetic moment are separated first in region 41′ due to stronger magnetic force, while smaller MNPs with lower magnetic moment are separated later in region 42′.

### Methods of Use

With the advantage of enhanced magnetic field as well as field gradient, there are numerous methods to utilize preferred embodiments for magnetic cell separation. Several examples are described herein.

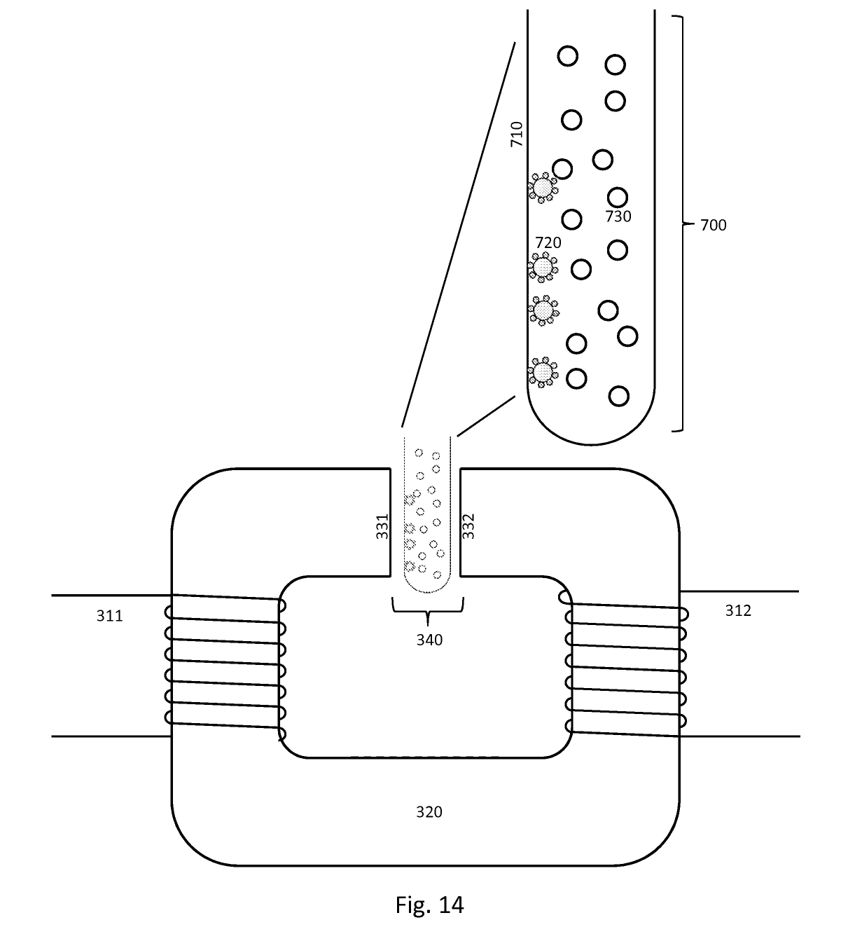

FIG. 14 illustrates one method of use for magnetic cell separation using preferred embodiments. MNPs conjugated with specific antibodies for rare target cells are first mixed with specimen such as whole blood in a container 710. After incubation period, MNPs conjugated with antibodies are bonded to surface of target cells, forming 720. Remaining non-target cells 730 are not bonded with MNPs and free floating. The mixture 700 of specimen in study and MNPs in the container 710 is then placed in an applied magnetic field generated by one of preferred embodiments as already described, such as Embodiment #1 for the purpose of illustration. The magnetic field is concentrated in the open gap 340 between pole tips 331/332. Depending on magnetic properties of MNPs, the apparatus which generates the magnetic field can operate in either Mode 1 for enhanced magnetic field strength or Mode 2 for enhanced field gradient. Target cells bonded with MNPs are attracted to the inner wall of the container 710 in the presence of an applied magnetic field. Non-target cells are then removed by transferring supernatant away. Target cells remain in the container 710 and can be then detached by physically removing the field generating apparatus away from the container, or powering off electrical current to the electromagnet. The collection of separated target cells can then be used for downstream analysis, such as staining, automatic counting, and DNA/RNA extraction, etc. This type of rare cell separation with target cells enriched and non-target cells removed is referred to as positive cell selection.

It should be noted that, while FIG. 14 shows the electromagnet embodiment as illustrated in FIG. 4, other preferred embodiments as well as alterations and modification as fall within the true spirit and scope of the present invention can be used.

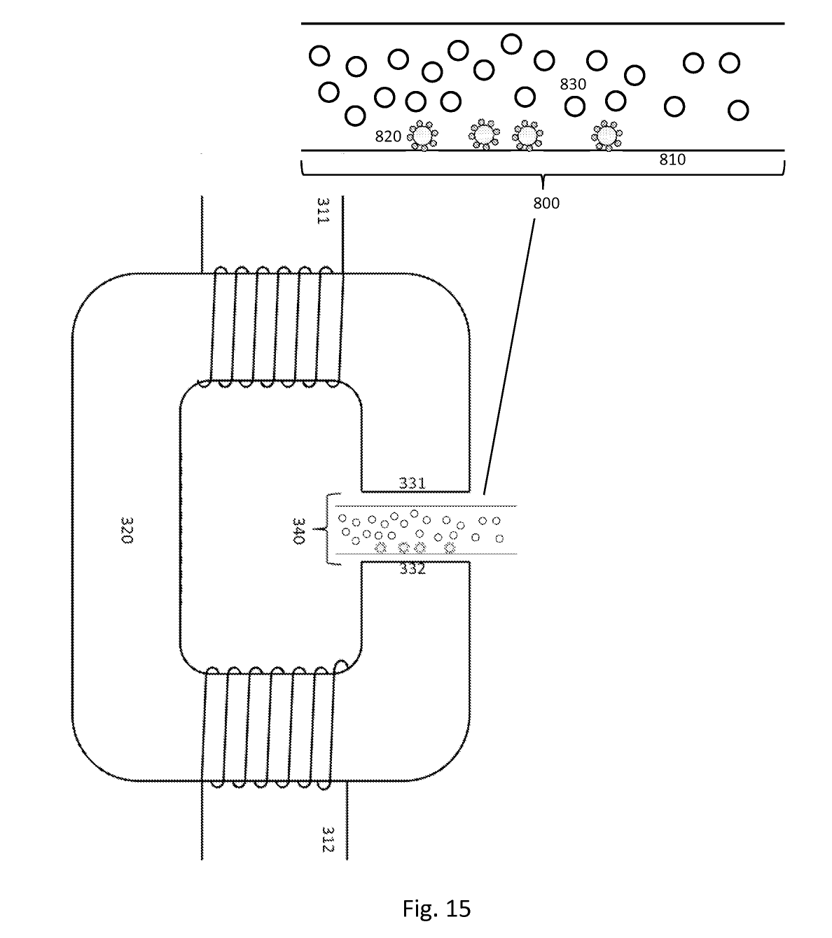

FIG. 15 illustrates another method of use for magnetic cell separation using preferred embodiments. MNPs conjugated with specific antibodies for rare target cells are first mixed. After incubation period, MNPs conjugated with antibodies are bonded to surface of target cells, forming 820. Remaining non-target cells 830 are not bonded with MNPs. The mixture is then flowed through a fluidic channel 810 which is placed in an applied magnetic field with the magnetic field direction perpendicular to the flow direction. The magnetic field is concentrated in the open gap 340 between pole tips 331/332. Target cells bonded with MNPs are attracted to the wall of the fluidic channel 710. The portion of the specimen which passes through the fluidic channel 710 therefore contains non-target cells only. This type of rare cell separation with target cells removed is also referred to as negative cell selection. As described earlier, spatial uniformity of the magnetic field and field gradient is advantageously improved with preferred embodiments. In another word, usable space with sufficient magnetic field and field gradient is enlarged. It is therefore feasible to use a plurality of fluidic channels within the open gap 340 and advantageously improve throughput and operation efficiency.

Again, it should be noted that, while FIG. 15 shows the electromagnet embodiment as illustrated in FIG. 4, other preferred embodiments as well as alterations and modification as fall within the true spirit and scope of the invention can be used.

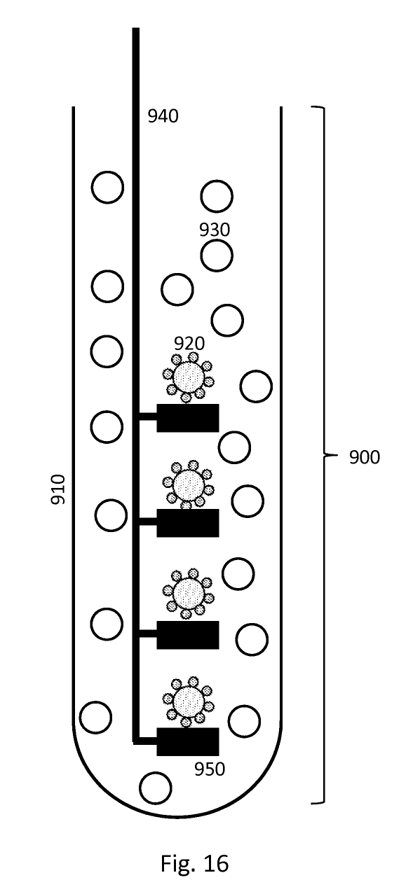

FIG. 16 illustrates yet another method of use with preferred embodiments. Instead of placing preferred embodiments outside specimen in container 710 as shown in FIG. 14 or in fluidic channel 810 as shown in FIG. 15, preferred embodiments can be miniaturized and directly placed inside the specimen for study, enabling direct in-vitro applications. A preferred analysis sequence is described herein. MNPs conjugated with specific antibodies for rare target cells are first mixed in container 910. After incubation period, MNPs conjugated with antibodies are bonded to surface of target cells, forming 920. Remaining non-target cells 930 are not bonded with MNPs. The magnetic field is generated by a plurality of miniaturized embodiments 950 powered by electrical currents distributed by a common electrical path 940, forming a bio-chip for in-vitro magnetic cell separation. A significant advantage is that target cells bonded with MNPs can be captured and are in direct contact with pole tips of preferred embodiments, thus avoiding the spacing loss associated with the thickness of the container wall or the fluidic channel. In comparison, the dimension of container thickness is on the order of hundreds of microns, while the dimension of MNPs is can be as small as of tens to hundreds of nanometers. This further enhances magnetic field strength as well as field gradient, and therefore separation efficiency.

It should be pointed out that the magnetic field profile generated by miniaturized embodiments is scale invariant due to the nature of magneto-static field. In another word, preferred embodiments with scaled-down dimension inherit the same field profile versus spacing from pole tip surface when normalized to dimension of pole tips. Miniaturized embodiments can be manufactured using nano-fabrication techniques and thin film processes which are widely adopted in semiconductor industry and hard disk drive (HDD) industry.

Although the present invention has been described in terms of specific embodiments, it is anticipated that alterations and modifications thereof will no doubt become apparent to those more skilled in the art. It is therefore intended that the following claims be interpreted as covering all such alterations and modification as fall within the true spirit and scope of the invention.

### REFERENCES

1. Recktenwald, D. and A. Radbruch, Cell Separation Methods and Applications, Marcel Dekker Inc., New York, 1997.
2. Portet, D., B. Denizot, E. Rump, J.-J. Lejeune and P. Jallet, Nonpolymeric Coatings of Iron Oxide Colloids for Biological Use as Magnetic Resonance Imaging Contrast Agents, J. Colloid Interface Sci., 238, pp. 37-42, 2001.
3. Butter, K., P. H. H. Bomans, P. M. Frederik, G. J. Vroege and A. P. Philipse, Direct observation of dipolar chains in iron ferrofluids by cryogenic electron microscopy, Nat. Mater., 2, pp. 88-91, 2003.
4. Cristofanilli, M. et al (2004): Circulating Tumor Cells, Disease Progression, and Survival in Metastatic Breast Cancer. NEJM 351:781-791.
5. Talasaz, A H et al (2009): Isolating Highly Enriched Populations of Circulating Epithelial Cells and Other Rare Cells from Blood Using a Magnetic Sweeper Device. PNAS USA 106: 3970-75.
6. Carpino, F., L. R. Moore, J. J. Chalmers, M. Zborowski and P. S. Williams, Quadrupole magnetic field-flow fractionation for the analysis of magnetic nanoparticles, J. Phys.: Conf. Ser., 17, pp. 174-180, 2005.

What is claimed is:

1. A magnetic field source to generate magnetic field for magnetic cell separation applications, comprising:
a magnet or a plurality of magnets;
at least one magnetic core;
a first pole tip and a second pole tip;
an open gap formed between said first pole tip and said second pole tip;
wherein,
a first magnetic field emanates from said first pole tip;
a second magnetic field emanates from said second pole tip;
said first magnetic field and second magnetic field are independently controlled.

2. The magnetic field source, as recited in claim 1, wherein magnetic field gradient and spatial uniformity is advantageously enhanced when said first and second magnetic fields are in opposite direction and preferably with different magnitude.

3. The magnetic field source, as recited in claim 1, wherein magnetic field strength and spatial uniformity is advantageously enhanced when said first and second magnetic fields are in the same direction with substantially comparable magnitude.

4. The magnetic field source, as recited in claim 1, is an electromagnet further comprising a first driving coil and an independently controlled second driving coil, wherein magnetic field magnitude and direction is controlled by electrical current magnitude and direction flowing through said first and second driving coils.

5. The electromagnet, as recited in claim 4, is operated to enhance magnetic field gradient and spatial uniformity in operation modes as recited in claim 2.

6. The electromagnet, as recited in claim 4, wherein magnetic core has permeability of greater than 500, and pole tips have magnetization greater than 0.5 Tesla.

7. The electromagnet, as recited in claim 4, further comprising a multi-section and reconfigurable magnetic core.

8. The magnetic field source, as recited in claim 1, has a hybrid configuration comprising an electromagnet and a permanent magnet, wherein an open gap is formed between pole tip of said electromagnet and pole tip of said permanent magnet.

9. The magnetic field source, as recited in claim 8, is operated to enhance magnetic field gradient and spatial uniformity in operation modes as recited in claim 2.

10. The magnetic field source, as recited in claim 1, comprising a first permanent magnet and a second permanent magnet, wherein an open gap is formed between pole tip of said first permanent magnet and pole tip of said second permanent magnet.

11. The magnetic field source, as recited in claim 10, is operated to enhance magnetic field gradient and spatial uniformity, in operation modes as recited in claim 2.

12. The magnetic field source, as recited in claim 1, comprising a first electromagnet and a second electromagnet, wherein an open gap is formed between pole tips of said first and second electromagnet.

13. The magnetic field source, as recited in claim 12, is operated to enhance magnetic field gradient and spatial uniformity, in operation modes as recited in claim 2.

14. The magnetic field source, as recited in claim 1, further comprising a magnetic core with tapered sections, wherein cross section area of said tapered sections shrinks towards pole tips to advantageously enhance magnetic field and field gradient.

15. The magnetic field source, as recited in claim 14, wherein said tapered sections further consist of magnetic materials with higher magnetization than that of the main magnetic cores.

16. A method of magnetic cell separation, comprising:
mixing specimen for analysis with MNPs conjugated with specific antibodies for rare target cells;
said MNPs conjugated with specific antibodies are bonded to surface of target cells;
applying a magnetic field using a magnetic field source with two independently controlled pole tips to enhance magnetic field gradient and therefore magnetic separation force.

17. The method of magnetic cell separation, as recited in claim 16, wherein said specimen for analysis is held in container, and target cells are attracted to the inner wall of said container. Positive cell separation is achieved by removing non-target cells.

18. The method of magnetic cell separation, as recited in claim 16, wherein specimen for analysis flows through a fluidic channel or a plurality of fluidic channels, and target cells are attracted to the wall of fluidic channels. Negative cell separation is achieved by collecting non-target cells which pass through.

19. The method of magnetic cell separation, as recited in claim 16, wherein said magnetic field source is miniaturized with magnetic field applied in-vitro to minimize spacing loss of magnetic field gradient and enhance separation effectiveness.

20. The method of magnetic cell separation, as recited in claim 16, wherein a plurality of said miniaturized magnetic field source with electrical currents distributed by a common electrical path form a bio-chip for in-vitro separation. A significant advantage is that target cells bonded with MNPs are in direct contact with pole tips of said miniaturized magnetic field source, thus avoiding the spacing loss associated with the thickness of the container wall or the fluidic channel.
